(19) United States
(12) Patent Application Publication
(10) Pub. No.: US 2026/0096975 A1
(43) Pub. Date: Apr. 9, 2026

(54) ACTIVE INGREDIENT COMBINATIONS OF ALKYLAMIDOTHIAZOLES AND ONE OR MORE BIOPOLYMERS

(71) Applicant: BEIERSDORF AG, Hamburg (DE)

(72) Inventors: Melina MEYER, Hamburg (DE); Caroline PLEHN, Hamburg (DE); Uta MEIRING, Hamburg (DE); Aylin STUHR, Hamburg (DE); Sylke HANSSON, Norderstedt (DE)

(21) Appl. No.: 19/108,523

(22) PCT Filed: Sep. 5, 2023
(86) PCT No.: PCT/EP2023/074256

§ 371 Date: Mar. 4, 2025

(30) Foreign Application Priority Data
Sep. 21, 2022 (DE) 10 2022 209 937.0

(51) Int. Cl. A61K 8/49; A61Q 19/02

(52) U.S. Cl. CPC A61K 8/49; A61Q 19/02

(57) ABSTRACT

Active ingredient combinations of one or more alkylamidothiazoles and one or more cosmetically or dermatologically acceptable biopolymers suitable for increasing the viscosity of an aqueous solution, selected from the group of the polysaccharides esterified and/or etherified with the alcohols provided with one or more hydroxyl groups and/or carboxylic acids and the unesterified and unetherified polysaccharides that are composed of interlinked glucose and/or mannose and/or galactose and/or glucuronic acid and/or rhamnose units, wherein, if xanthan or hydroxypropyl starch phosphate is selected as biopolymer, at least one further biopolymer must be present.

The present invention relates to active ingredient combinations of one or more alkylamidothiazoles and one or more biopolymers. Furthermore, the present invention relates to cosmetic or dermatological preparations having a content of such active ingredient combination, and to the use thereof for lightening human skin.

Melanocytes are responsible for the pigmentation of the skin; these are found in the lowermost layer of the epidermis, the stratum basale, next to the basal cells, as pigment-forming cells that occur either individually or in clusters of varying size, depending on the skin type.

As characteristic cell organelles, melanocytes contain melanosomes, in which melanin is formed. Melanin formation is increased inter alia upon excitation by UV radiation. This melanin is transported via the living layers of the epidermis (keratinocytes) ultimately into the horny layer (corneocytes) and elicits a more or less pronounced brownish to brown-black skin color.

Melanin is formed as the final stage of an oxidative process in which tyrosine is converted, with the assistance of the enzyme tyrosinase, via several intermediate stages to the brown to brown-black eumelanins (DHICA and DHI melanin), or, with the involvement of sulfur-containing compounds, to the reddish pheomelanin. DHICA and DHI melanin are formed via the common intermediate stages dopaquinone and dopachrome. The latter, in part with the involvement of further enzymes, is converted either to indole-5,6-quinone carboxylic acid or to indole-5,6-quinone, from which the two specified eumelanins are formed.

Pheomelanin is formed inter alia via the intermediate products dopaquinone and cysteinyldopa. The expression of the melanin-synthesizing enzymes is controlled by a specific transcription factor (microphthalmia-associated transcription factor, MITF). In addition to the described enzymatic processes of melanin synthesis, melanosomes also contain further proteins that are significant to melanogenesis. The so-called P protein appears to be assigned an important role here, but the exact function is still unclear.

In addition to the above-described process of melanin synthesis in melanocytes, the transfer of melanosomes, their stay in the epidermis and also their degradation and the degradation of melanin are also of crucial importance for the pigmentation of the skin. It was able to be demonstrated that the PAR-2 receptor is significant for the transport of melanosomes from melanocytes into keratinocytes (M. Seiberg et al., 2000, J. Cell. Sci., 113:3093-101).

Furthermore, the size and shape of melanosomes have an influence on their light-scattering properties and thus the appearance of the skin in terms of color. For example, in sub-Saharan Africans there are greater numbers of large spheroidal melanosomes that occur individually, while in Caucasians melanosomes are found that are rather smaller and occur in groups.

Problems with hyperpigmentation of the skin have a wide variety of causes, or are accompanying phenomena of many biological processes, e.g. UV radiation (e.g. freckles, Ephelides), genetic predisposition, incorrect pigmentation of the skin during wound healing or scarring (post-inflammatory hyperpigmentation), or skin aging (e.g. Lentigines seniles).

After inflammatory responses, the pigmentation system of the skin reacts with partially contradictory responses. This can lead to both post-inflammatory hyper- and hypopigmentation. Post-inflammatory hypomelanoses commonly occur inter alia in conjunction with atopy, lupus erythematosus and psoriasis. The different forms of responses of the pigmentation system of the human skin as a consequence of inflammatory phenomena are understood only very poorly.

Problems with post-inflammatory hyperpigmentation commonly occur in darker skin types. The problem of Pseudofollikulitis barbae, which is associated with cosmetically undesirable incorrect pigmentation or gives rise to same, is known especially in male people of color. Forms of melasma, which occur especially in women of Asian origin on the face and on the décolletage area, and also various forms of irregular pigmentation of the skin, are also included among the occurrences of post-inflammatory hyperpigmentation. Furthermore, dark circles around the eyes are also considered as a form of post-inflammatory hyperpigmentation, with the underlying inflammation usually occurring on a subclinical level.

In many cases, such post-inflammatory incorrect pigmentation is further increased by the action of sunlight (UV light), without UV-induced inflammation (sunburn) arising.

Active ingredients and preparations which counteract skin pigmentation are known. In practical use are essentially preparations based on hydroquinone, although, on the one hand, these only exhibit their effect after use for several weeks, and, on the other hand, their excessively long use is ill-advised for toxicological reasons. Albert Kligman et al. developed a so-called triformula, which is a combination of 0.1% tretinoin, 5.0% hydroquinone, 0.1% dexamethasone (A. Kligman, 1975, Arch. Dermatol., 111:40-48). However, this formulation is also highly controversial due to possible irreversible changes in the pigmentation system of the skin.

Furthermore, skin-peeling methods (chemical and mechanical peels) are used, however they often give rise to inflammatory reactions and can even, due to post-inflammatory hyperpigmentation which occurs thereafter, lead to increased, instead of decreased, pigmentation. All of these customary methods, which are also used for treating post-inflammatory hyperpigmentation, are characterized by critical side effects.

Further known are various other substances for which skin-lightening efficacy is described. Mention is to be made here inter alia of hexadecene-1,16-dicarboxylic acid, kojic acid and derivatives, arbutin, ascorbic acid and derivatives, flavonoids, ellagic acid and derivatives, tranexamic acid and various resorcinol derivatives, such as 4-n-butylresorcinol, 4-n-hexylresorcinol and 4-(1-phenylethyl)benzene-1,3-diol.

In a publication (Bioorganic & Medicinal Chemistry Letter 17 (2007) 6871-6875, J. M. Ready describes the action of inter alia substituted thiazole derivatives for the inhibition of mushroom tyrosinase.

The patent application from Shiseido (WO 2009099195) describes substituted thiazolamines or hydrothiazolamines for skin lightening.

The substances described in the above-mentioned prior art exhibit moderate efficacy.

Circles around the eyes may likewise arise as a consequence of a pigmentation disorder, but they may also appear as a response to general stress, such as a lack of sleep or simply by excessive eye strain. In younger people, the symptoms disappear again after sufficient sleep, however over prolonged periods the condition may become chronic and very bothersome for the afflicted persons. There is also a lack of sufficiently promising active ingredients and treatment options for such skin phenomena.

Smell is one of the five human senses. This involves odorous substances, highly volatile substances, being bound to olfactory receptors in the nasal mucosa. Via a signaling cascade, the stimulus is transmitted to the nerve cells and passed on to the brain. This is where the actual sense of smell arises. Pleasant odors are distinguished from unpleasant odors.

However, cosmetic products often also have a slightly unpleasant inherent odor that is caused by the raw materials used. Alkylamidothiazoles can under some circumstances also contribute to a deterioration in the fragrance experience of a cosmetic base. The aim was to remedy this shortcoming.

This object is achieved by active ingredient combinations of one or more alkylamidothiazoles and one or more cosmetically or dermatologically acceptable biopolymers suitable for increasing the viscosity of an aqueous solution, selected from the group of the polysaccharides esterified and/or etherified with alcohols provided with one or more hydroxyl groups and/or carboxylic acids and the unesterified and unetherified polysaccharides that are composed of interlinked glucose and/or mannose and/or galactose and/or glucuronic acid and/or rhamnose units, wherein, if xanthan or hydroxypropyl starch phosphate is selected as biopolymer, at least one further biopolymer must be present.

Preferably, the preparations according to the invention contain 0.0001%-10% by weight of one or more biopolymers, preferably 0.001%-5% by weight of one or more biopolymers, especially preferably 0.005%-3% by weight of one or more biopolymers.

Particularly advantageous are preparations or uses according to the invention, characterized in that the preparations contain 0.000001% to 10% by weight, in particular 0.0001% to 3% by weight, very particularly 0.001% to 1% by weight of one or more alkylamidothiazoles, based on the total weight of the preparation.

Advantageous alkylamidothiazoles in the context of the present invention are substances of the general formula

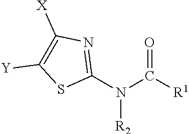

in which
R¹, R², X and Y may be different, partially identical or entirely identical and may independently have the following meanings:
R¹=—C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkylhydroxy (linear and branched), —C₁-C₂₄ alkylamine (linear and branched), —C₁-C₂₄ alkylaryl (linear and branched), —C₁-C₂₄ alkylaryl-alkyl-hydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkyl-morpholino, —C₁-C₂₄ alkyl-piperidino, —C₁-C₂₄ alkyl-piperazino, —C₁-C₂₄ alkyl-piperazino-N-alkyl,
R²=H, —C₁-C₂₄ alkyl (linear and branched), —C₁-C₂₄ alkenyl (linear and branched), —C₁-C₈ cycloalkyl, —C₁-C₂₄ hydroxyalkyl (linear and branched), —C₁-C₂₄ alkylaryl (linear and branched), —C₁-C₂₄ alkylheteroaryl (linear and branched),
X=—H, —C₁-C₂₄ alkyl (linear and branched), —C₁-C₂₄ alkenyl (linear and branched), —C₁-C₈ cycloalkyl, —C₁-C₂₄ aryl (optionally mono- or polysubstituted by —OH, —F, —Cl, —Br, —I, —OMe, —NH₂, —CN), —C₁-C₂₄ heteroaryl (optionally mono- or polysubstituted by —OH, —F, —Cl, —Br, —I, —OMe, —NH₂, —CN), —C₁-C₂₄ alkylaryl (linear and branched), —C₁-C₂₄ alkylheteroaryl (linear and branched), -aryl (optionally mono- or polysubstituted by —OH, —F, —Cl, —Br, —I, —OMe, —NH₂, —CN), -phenyl, -2,4-dihydroxyphenyl, -2,3-dihydroxyphenyl, -2,4-dimethoxyphenyl, -2,3-dimethoxyphenyl,
Y=H, —C₁-C₂₄ alkyl (linear and branched), —C₁-C₂₄ alkenyl (linear and branched), —C₁-C₈ cycloalkyl, —C₁-C₂₄ aryl, —C₁-C₂₄ heteroaryl, —C₁-C₂₄ alkylaryl (linear and branched), —C₁-C₂₄ alkylheteroaryl (linear and branched), -aryl, -phenyl, -2,4-dihydroxyphenyl, -2,3-dihydroxyphenyl, -2,4-dimethoxyphenyl, -2,3-dimethoxyphenyl, —COO-alkyl, —COO-alkenyl, —COO-cycloalkyl, —COO-aryl, —COO-heteroaryl,
and X, Y may optionally also=fused aromatic,
where X and Y may form, with one another, aromatic or aliphatic homo- or heterocyclic ring systems having up to n ring-forming atoms, and where the number n may assume values from 5 to 8, and the respective ring systems in turn may be substituted by up to n−1 alkyl groups, hydroxyl groups, carboxyl groups, amino groups, nitrile functions, sulfur-containing substituents, ester groups and/or ether groups.

The thiazoles mentioned may be present either as free base or as salt: e.g. as fluoride, chloride, bromide, iodide, sulfate, carbonate, ascorbate, acetate or phosphate. Especially as halogen salts, such as chloride and bromide.

Furthermore, an advantageous implementation of the present invention consists in cosmetic or dermatological preparations having an effective content of one or more aforementioned alkylamidothiazoles.

The use of the aforementioned alkylamidothiazoles for the treatment and/or prophylaxis of undesired skin pigmentation is also according to the invention. In this case, treatment and/or prophylaxis of undesired skin pigmentation may be both in the cosmetic and the pharmaceutical domain.

In this case, the pharmaceutical (or dermatological) treatment is primarily understood in terms of diseased skin conditions, whereas the cosmetic treatment and/or prophylaxis of undesired skin pigmentation primarily relates to healthy skin.

Advantageously, X is selected from the group of substituted phenyls, where the substituents (Z) may be selected from the group of —H, —OH, —F, —Cl, —Br, —I, —OMe, —NH₂, —CN, acetyl and may be identical or different.

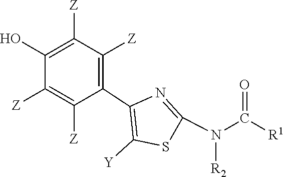

Particularly advantageously, X is selected from the group of phenyl groups substituted by one or more hydroxy groups, where the substituent (Z) may be selected from the group of —H, —OH, —F, —Cl, —Br, —I, —OMe, —NH₂, —CN, acetyl and preference is given to the following generic structure, in which Y, R¹ and R² may have the properties defined above.

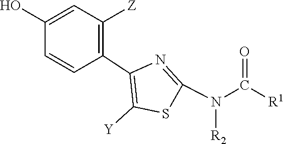

Especially advantageous are those compounds in which

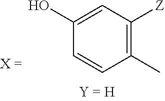

R₁=—C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkylhydroxy (linear and branched), —C₁-C₂₄ alkylamine (linear and branched), —C₁-C₂₄ alkylaryl (linear and branched), —C₁-C₂₄ alkylaryl-alkyl-hydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkyl-morpholino, —C₁-C₂₄ alkyl-piperidino, —C₁-C₂₄ alkyl-piperazino, —C₁-C₂₄ alkyl-piperazino-N-alkyl,
R²=H, —C₁-C₂₄ alkyl (linear and branched),
Z=−H, —OH, —F, —Cl, —Br, —I, —OMe, —NH₂, —CN, acetyl.

Particular preference is given to those compounds in which

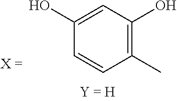

R₁=—C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkylhydroxy (linear and branched), —C₁-C₂₄ alkylamine (linear and branched), —C₁-C₂₄ alkylaryl (linear and branched), —C₁-C₂₄ alkylaryl-alkyl-hydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkyl-morpholino, —C₁-C₂₄ alkyl-piperidino, —C₁-C₂₄ alkyl-piperazino, —C₁-C₂₄ alkyl-piperazino-N-alkyl,
R²=H.

The compounds

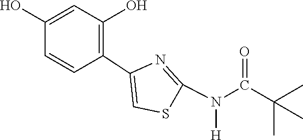 [SMILES: CC(C)(C)C(=O)Nc1nc(-c2ccc(O)cc2O)cs1]

### N-(4-(2,4-dihydroxyphenyl)thiazol-2-yl)pivalamide

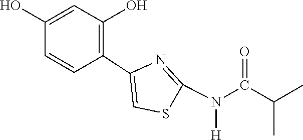 [SMILES: CC(C)C(=O)Nc1nc(-c2ccc(O)cc2O)cs1]

### N-(4-(2,4-dihydroxyphenyl)thiazol-2-yl)isobutyramide

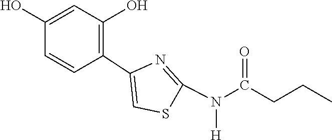 [SMILES: CCCC(=O)Nc1nc(-c2ccc(O)cc2O)cs1]

### N-(4-(2,4-dihydroxyphenyl)thiazol-2-yl)butyramide

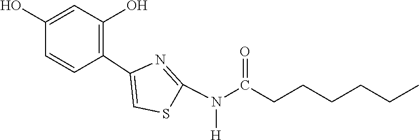 [SMILES: CCCCCCC(=O)Nc1nc(-c2ccc(O)cc2O)cs1]

### N-(4-(2,4-dihydroxyphenyl)thiazol-2-yl)heptanamide

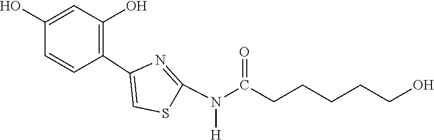 [SMILES: O=C(CCCCCO)Nc1nc(-c2ccc(O)cc2O)cs1]

### N-(4-(2,4-dihydroxyphenyl)thiazol-2-yl)-6-hydroxyhexanamide

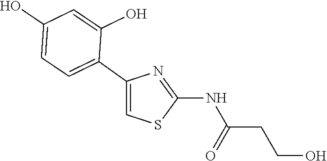 [SMILES: O=C(CCO)Nc1nc(-c2ccc(O)cc2O)cs1]

### N-(4-(2,4-dihydroxyphenyl)thiazol-2-yl)-3-hydroxypropanamide

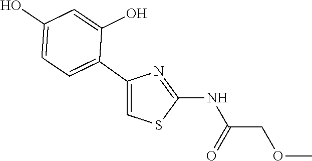 [SMILES: COCC(=O)Nc1nc(-c2ccc(O)cc2O)cs1]

### N-(4-(2,4-dihydroxyphenyl)thiazol-2-yl)-2-methoxyacetamide

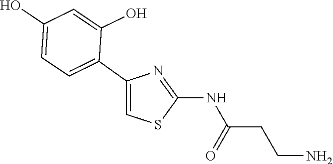 [SMILES: NCCC(=O)Nc1nc(-c2ccc(O)cc2O)cs1]

### 3-amino-N-(4-(2,4-dihydroxyphenyl)thiazol-2-yl)propanamide

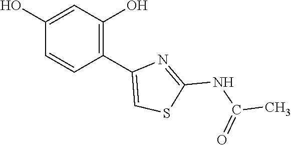 [SMILES: CC(=O)Nc1nc(-c2ccc(O)cc2O)cs1]

### N-(4-(2,4-dihydroxyphenyl)thiazol-2-yl)acetamide

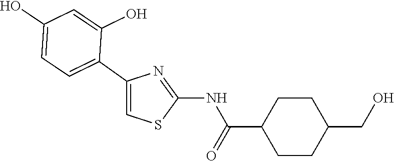 [SMILES: O=C(Nc1nc(-c2ccc(O)cc2O)cs1)C1CCC(CO)CC1]

### N-(4-(2,4-dihydroxyphenyl)thiazol-2-yl)-4-(hydroxymethyl)cyclohexanecarboxamide

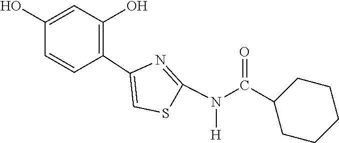 [SMILES: O=C(Nc1nc(-c2ccc(O)cc2O)cs1)C1CCCCC1]

### N-(4-(2,4-dihydroxyphenyl)thiazol-2-yl)cyclohexanecarboxamide

and

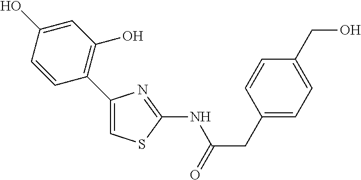 [SMILES: O=C(Cc1ccc(CO)cc1)Nc1nc(-c2ccc(O)cc2O)cs1]

### N-(4-(2,4-dihydroxyphenyl)thiazol-2-yl)-2-(4-(hydroxymethyl)phenyl)acetamide

are those preferred according to the invention.

Very particular preference is given to

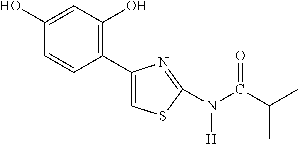 [SMILES: CC(C)C(=O)Nc1nc(-c2ccc(O)cc2O)cs1]

### N-(4-(2,4-dihydroxyphenyl)thiazol-2-yl)isobutyramide

The above alkylamidothiazoles, their synthesis and use are described in EP2758381A1.

Biopolymers preferred according to the invention include dehydroxanthan, gellan (INCI: Gellan Gum), xanthan, hydroxypropyl starch phosphate (INCI: Hydroxypropyl Starch Phosphate) and any mixtures thereof.

Particularly advantageous in the context of the present invention are mixtures of
a) dehydroxanthan,
b) gellan,
c) xanthan,
d) hydroxypropyl starch phosphate,
the a:b, a:c, a:d, b:c, b:d and c:d weight ratios of which each independently behave like rational numbers between 0 and 10.

Very particularly advantageous in the context of the present invention are mixtures of
a) dehydroxanthan,
b) gellan,
c) xanthan,
d) hydroxypropyl starch phosphate,
the a:b:c:d weight ratios of which behave in relation to one another as follows: (0.1 to 5):(0.1 to 5):(0.1 to 5):(0.1 to 5).

It may be advantageous to dispense with other substances that increase the viscosity of aqueous solutions (“thickeners”). In particular, it is advantageous to dispense with polyacrylates that have the effect of increasing the viscosity of aqueous solutions.

Cosmetic or dermatological preparations having a content of alkylamidothiazoles and biopolymers according to the invention or the use thereof for the treatment and/or prophylaxis of undesired skin pigmentation are likewise advantageous embodiments of the present invention.

It is especially advantageous if such preparations contain 0.000001% to 10% by weight, in particular 0.0001% to 3% by weight, very particularly 0.001% to 1% by weight of one or more of the alkylamidothiazoles used according to the invention, based on the total weight of the preparation.

It is highly advantageous in the context of the present invention if the preparations have slightly basic pHs, i.e. values between 7 and 9, preferably between 7 and 8, in particular between 7.4 and 7.6.

Cosmetic and dermatological preparations according to the invention may be present in different forms. For example, they may be a solution, an anhydrous preparation, an emulsion or microemulsion of water-in-oil (W/O) type or of oil-in-water (O/W) type, a multiple emulsion, for example of water-in-oil-in-water (W/O/W) type, a gel, a solid stick, an ointment or else an aerosol. It is also advantageous according to the invention to administer the substances used according to the invention and/or the derivatives thereof in encapsulated form, e.g. in collagen matrices and other customary encapsulation materials, e.g. as cellulose encapsulations, in gelatin or liposomally encapsulated.

It is also possible and advantageous in the context of the present invention to add the substances used according to the invention and/or the derivatives thereof to aqueous systems or surfactant preparations for cleansing the skin and the hair.

The cosmetic and dermatological preparations according to the invention may comprise cosmetic auxiliaries, such as those usually used in such preparations, for example preservatives, bactericides, perfumes, substances for preventing foaming, dyes, pigments that have a coloring effect, thickeners, surface-active substances, emulsifiers, softening, moistening and/or moisturizing substances, fats, oils, waxes or other customary constituents of a cosmetic or dermatological formulation such as alcohols, polyols, polymers, foam stabilizers, electrolytes, organic solvents or silicone derivatives.

The lipid phase may advantageously be selected from the following group of substances:
mineral oils, mineral waxes;
oils, such as triglycerides of capric or caprylic acid, also natural oils such as castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils such as dimethylpolysiloxanes, diethylpolysiloxanes, diphenylpolysiloxanes, and mixed forms thereof.

The oil phase of the emulsions, oleogels or hydrodispersions or lipodispersions in the context of the present invention is advantageously selected from the group of the esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, from the group of the esters of aromatic carboxylic acids and saturated and/or unsaturated, branched and/or unbranched alcohols having a chain length of 3 to 30 carbon atoms. Such ester oils may then advantageously be selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, Dibutyl Adipate, Propylheptyl Caprylate, Diisopropyl Adipate, Cetearyl Isononanoate, oleyl erucate, erucyl oleate, erucyl erucate, and synthetic, semisynthetic and natural mixtures of such esters, for example jojoba oil.

The aqueous phase of the preparations according to the invention optionally advantageously contains humectants such as propylene glycol, panthenol or hyaluronic acid, in each case individually or in combination.

In particular, use is made of mixtures of the above-mentioned solvents. In the case of alcoholic solvents, water may be a further constituent.

Emulsions according to the invention are advantageous and contain for example the mentioned fats, oils, waxes and other fatty substances, and water and an emulsifier, such as is usually used for such a type of formulation.

Gels according to the invention usually contain alcohols of low carbon number, for example ethanol, propylene glycol, and water or an above-mentioned oil in the presence of a thickener. Suitable as propellant for preparations according to the invention that are sprayable from aerosol containers are the highly volatile, liquefied propellants that are customary and known, for example hydrocarbons (propane, butane, isobutane), which may be used alone or in a mixture with one another. Compressed air may also advantageously be used.

Advantageously, preparations according to the invention may further contain substances that absorb UV radiation in the UVB range, where the total amount of the filter substances is for example 0.1% to 30% by weight, preferably 0.5% to 10% by weight, in particular 1.0% to 6.0% by weight, based on the total weight of the preparations, in order to provide cosmetic preparations that protect the hair or the skin from the entire range of ultraviolet radiation. They may also be used as sunscreens for the hair or the skin.

Moreover, preparations according to the invention may advantageously additionally contain substances that conceal the unpleasant intrinsic odor of the rest of the raw materials used, where the total amount of the perfume ingredients is for example 0.001% by weight to 30% by weight, preferably 0.05% to 10% by weight, in particular 0.1% to 5.0% by weight, based on the total weight of the preparations, in order to provide cosmetic preparations.

### EXPERIMENTAL DESCRIPTION

To determine the influence of the biopolymers used in the formula on the odor development in combination with the active ingredient N-(4-(2,4-dihydroxyphenyl)thiazol-2-yl)isobutyramide in comparison with the synthetic polymer sodium polyacrylate (INCI: Sodium Polyacrylate), the samples were irradiated for 48 h using a so-called Atlas Suntester. All samples were in 30 ml glass sample vials. The irradiance of the device was 250 W/m², which after an irradiation time of 48 h corresponds to storage of the samples after 8 months in a light window. The samples were then assessed unopened by a trained panel of fragrance experts to assess the odor development and odor intensity. In order to establish comparability of the batches, all further odor-influencing constituents of the formulation were removed or used in the same concentration. In this specific case, this means that all formulations are perfume-free and have the same ethanol concentration.

| Ingredients (INCI) | Inv. 1 | Inv. 2 | Inv. 3 |
|---|---|---|---|
| Sodium Hyaluronate | 0.20 | 0.58 | 0.20 |
| Isobutylamido | 0.18 | 0.18 | 0.18 |
| Thiazolyl Resorcinol | | | |
| Propylheptyl | 2.00 | 0 | 2.00 |
| Caprylate | | | |
| Isopropyl Palmitate | 2.50 | 2.50 | 2.50 |
| Diisopropyl Adipate | 2.00 | 0 | 2.00 |
| Cetearyl | 2.00 | 2.00 | 2.00 |
| Isononanoate | | | |
| Sodium Stearoyl | 0.20 | 0.10 | 0.20 |
| Glutamate | | | |
| Glyceryl Stearate | 0.75 | 0.25 | 0.75 |
| Glycerol | 5.00 | 5.00 | 5.00 |
| Methylpropanediol | 0 | 1.00 | 0 |
| Butylene Glycol | 5.00 | 5.00 | 5.00 |
| Caprylyl Glycol | 0.15 | 0.13 | 0.15 |
| Citric Acid | 0.04 | 0.027 | 0.04 |
| Phenoxyethanol | 0.50 | 0.20 | 0.50 |
| Aqua | to 100 | to 100 | to 100 |
| Alcohol Denat. | 6.00 | 6.00 | 6.00 |
| Trisodium EDTA | 1.00 | 0.50 | 1.00 |
| Octocrylene | 0.00 | 4.00 | 0.00 |
| Cetearyl Alcohol | 1.75 | 0 | 1.75 |
| Dehydroxanthan | 0.20 | 0 | 0.20 |
| Gum | | | |
| Gellan Gum | 0.05 | 0 | 0 |
| Xanthan Gum | 0.10 | 0 | 0 |
| Hydroxypropyl | 0.75 | 0 | 0 |
| Starch Phosphate | | | |
| Sodium Polyacrylate | 0 | 0.35 | 0 |
| Observation | less odor | more odor | lowest odor |
| | development in the | development in the | development by |
| | case of Inv. 1 in | case of Inv. 2 in | Inv. 3 in comparison |
| | comparison with | comparison with | with Inv. 4, 5 & 6 |
| | Inv. 2; | Inv. 1 | |
| | second-lowest odor | | |
| | development in | | |
| | comparison with | | |
| | Inv. 7, 8 & 9 (pH of | | |
| | the preparation | | |
| | pH = 6) | | |
| Ingredients (INCI) | Inv. 4 | Inv. 5 | Inv. 6 |
| Sodium Hyaluronate | 0.20 | 0.20 | 0.20 |
| Isobutylamido | 0.18 | 0.18 | 0.18 |
| Thiazolyl Resorcinol | | | |
| Propylheptyl | 2.00 | 2.00 | 2.00 |
| Caprylate | | | |
| Isopropyl Palmitate | 2.50 | 2.50 | 2.50 |
| Diisopropyl Adipate | 2.00 | 2.00 | 2.00 |
| Cetearyl | 2.00 | 2.00 | 2.00 |
| Isononanoate | | | |
| Sodium Stearoyl | 0.20 | 0.20 | 0.20 |
| Glutamate | | | |
| Glyceryl Stearate | 0.75 | 0.75 | 0.75 |
| Glycerol | 5.00 | 5.00 | 5.00 |
| Butylene Glycol | 5.00 | 5.00 | 5.00 |
| Caprylyl Glycol | 0.15 | 0.15 | 0.15 |
| Citric Acid | 0.04 | 0.04 | 0.04 |
| Phenoxyethanol | 0.50 | 0.50 | 0.50 |
| Aqua | to 100 | to 100 | to 100 |
| Alcohol Denat. | 6.00 | 6.00 | 6.00 |
| Trisodium EDTA | 1.00 | 1.00 | 1.00 |
| Cetearyl Alcohol | 1.75 | 1.75 | 1.75 |
| Dehydroxanthan | 0 | 0 | 0 |
| Gum | | | |
| Gellan Gum | 0.05 | 0 | 0 |
| Xanthan Gum | 0 | 0.10 | 0 |
| Hydroxypropyl | 0 | 0 | 0.75 |
| Starch Phosphate | | | |
| Observation | third-lowest odor | second-lowest odor | fourth-lowest odor |
| | development by | development by | development by |
| | Inv. 4 in comparison | Inv. 5 in comparison | Inv. 6 in comparison |
| | with Inv. 3, 5 & 6 | with Inv. 2, 4 & 6 | with Inv. 3, 4 & 5 |
| Ingredients (INCI) | Inv. 7 | Inv. 8 | Inv. 9 |
| Sodium Hyaluronate | 0.20 | 0.20 | 0.20 |
| Isobutylamido | 0.18 | 0.18 | 0.18 |
| Thiazolyl Resorcinol | | | |
| Propylheptyl | 2.00 | 2.00 | 2.00 |
| Caprylate | | | |
| Isopropyl Palmitate | 2.50 | 2.50 | 2.50 |
| Diisopropyl Adipate | 2.00 | 2.00 | 2.00 |
| Cetearyl | 2.00 | 2.00 | 2.00 |
| Isononanoate | | | |
| Sodium Stearoyl | 0.20 | 0.20 | 0.20 |
| Glutamate | | | |
| Glyceryl Stearate | 0.75 | 0.75 | 0.75 |
| Glycerol | 5.00 | 5.00 | 5.00 |
| Butylene Glycol | 5.00 | 5.00 | 5.00 |
| Caprylyl Glycol | 0.15 | 0.15 | 0.15 |
| Citric Acid | 0.04 | 0.04 | 0.04 |
| Phenoxyethanol | 0.50 | 0.50 | 0.50 |
| Aqua | to 100 | to 100 | to 100 |
| Alcohol Denat. | 6.00 | 6.00 | 6.00 |
| Trisodium EDTA | 1.00 | 1.00 | 1.00 |
| Cetearyl Alcohol | 1.75 | 1.75 | 1.75 |
| Dehydroxanthan | 0.20 | 0.20 | 0.20 |
| Gum | | | |
| Gellan Gum | 0.05 | 0.05 | 0.05 |
| Xanthan Gum | 0.10 | 0.10 | 0.10 |
| Hydroxypropyl | 0.75 | 0.75 | 0.75 |
| Starch Phosphate | | | |
| Observation | fourth-lowest odor | third-lowest odor | lowest odor |
| | development by | development by | development by |
| | Inv. 7 in comparison | Inv. 8 in comparison | Inv. 9 in comparison |
| | with Inv. 1, 8 & 9 | with Inv. 1, 7 & 9 | with Inv. 1, 7 & 8 |
| | (pH of the | (pH of the | (pH of the |
| | preparation pH = 4.0) | preparation pH = 5.0) | preparation pH = 7.5) |

The examples below are intended to illustrate the present invention without limiting it. Unless stated otherwise, all amounts, proportions and percentages are based on the weight and the total amount or on the total weight of the preparations.

| Ingredients (INCI) | Example 1 | Example 2 | Example 3 |
|---|---|---|---|
| Sodium Hyaluronate | 0.20 | 0.20 | 0.20 |
| Isobutylamido | 0.18 | 0.18 | 0.18 |
| Thiazolyl Resorcinol | | | |
| Propylheptyl | 2.00 | 2.00 | 2.00 |
| Caprylate | | | |
| Isopropyl Palmitate | 2.50 | 2.50 | 2.50 |
| Diisopropyl Adipate | 2.00 | 2.00 | 2.00 |
| Cetearyl | 2.00 | 2.00 | 2.00 |
| Isononanoate | | | |
| Sodium Stearoyl | 0.20 | 0.20 | 0.20 |
| Glutamate | | | |
| Glyceryl Stearate | 0.75 | 0.75 | 0.75 |
| Glycerol | 5.00 | 5.00 | 5.00 |
| Butylene Glycol | 5.00 | 5.00 | 5.00 |
| Caprylyl Glycol | 0.15 | 0.15 | 0.15 |
| Citric Acid | 0.04 | 0.04 | 0.04 |
| Phenoxyethanol | 0.50 | 0.50 | 0.50 |
| Aqua | to 100 | to 100 | to 100 |
| Alcohol Denat. | 6.00 | 6.00 | 6.00 |
| Trisodium EDTA | 1.00 | 1.00 | 1.00 |
| Cetearyl Alcohol | 1.75 | 1.75 | 1.75 |
| Dehydroxanthan | 0.10 | 0 | 0.10 |
| Gum | | | |
| Gellan Gum | 0.10 | 0.05 | 0.10 |
| Xanthan Gum | 0.10 | 0.10 | 0.10 |
| Hydroxypropyl | 1.00 | 0.75 | 0.75 |
| Starch Phosphate | | | |
| Ingredients | Example 4 | Example 5 | Example 6 |
| Sodium Hyaluronate | 0.20 | 0.20 | 0.20 |
| Isobutylamido | 0.18 | 0.18 | 0.18 |
| Thiazolyl Resorcinol | | | |
| Propylheptyl | 2.00 | 2.00 | 2.00 |
| Caprylate | | | |
| Isopropyl Palmitate | 2.50 | 2.50 | 2.50 |
| Diisopropyl Adipate | 2.00 | 2.00 | 2.00 |
| Cetearyl | 2.00 | 2.00 | 2.00 |
| Isononanoate | | | |
| Sodium Stearoyl | 0.20 | 0.20 | 0.20 |
| Glutamate | | | |
| Glyceryl Stearate | 0.75 | 0.75 | 0.75 |
| Glycerol | 5.00 | 5.00 | 5.00 |
| Butylene Glycol | 5.00 | 5.00 | 5.00 |
| Caprylyl Glycol | 0.15 | 0.15 | 0.15 |
| Citric Acid | 0.04 | 0.04 | 0.04 |
| Phenoxyethanol | 0.50 | 0.50 | 0.50 |
| Aqua | to 100 | to 100 | to 100 |
| Alcohol Denat. | 6.00 | 6.00 | 6.00 |
| Trisodium EDTA | 1.00 | 1.00 | 1.00 |
| Cetearyl Alcohol | 1.75 | 1.75 | 1.75 |
| Dehydroxanthan | 0.20 | 0 | 0 |
| Gum | | | |
| Gellan Gum | 0.05 | 0.05 | 0.10 |
| Xanthan Gum | 0.10 | 0.10 | 0.10 |
| Hydroxypropyl | 0.75 | 0.75 | 0.75 |
| Starch Phosphate | | | |
| Perfume | 0.50 | 0 | 0 |
| Succinoglycan | 0 | 0.2 | 0 |
| Ingredients | Example 7 | Example 8 | Example 9 |
| Sodium Hyaluronate | 0.20 | 0.20 | 0.20 |
| Isobutylamido | 0.10 | 0.20 | 0.30 |
| Thiazolyl Resorcinol | | | |
| Propylheptyl | 2.00 | 2.00 | 2.00 |
| Caprylate | | | |
| Isopropyl Palmitate | 2.50 | 2.50 | 2.50 |
| Diisopropyl Adipate | 2.00 | 2.00 | 2.00 |
| Cetearyl | 2.00 | 2.00 | 2.00 |
| Isononanoate | | | |
| Sodium Stearoyl | 0.20 | 0.20 | 0.20 |
| Glutamate | | | |
| Glyceryl Stearate | 0.75 | 0.75 | 0.75 |
| Glycerol | 5.00 | 5.00 | 5.00 |
| Butylene Glycol | 5.00 | 5.00 | 5.00 |
| Caprylyl Glycol | 0.15 | 0.15 | 0.15 |
| Citric Acid | 0.04 | 0.04 | 0.04 |
| Phenoxyethanol | 0.50 | 0.50 | 0.50 |
| Aqua | to 100 | to 100 | to 100 |
| Alcohol Denat. | 6.00 | 6.00 | 6.00 |
| Trisodium EDTA | 1.00 | 1.00 | 1.00 |
| Cetearyl Alcohol | 1.75 | 1.75 | 1.75 |
| Dehydroxanthan | 0.20 | 0.20 | 0.20 |
| Gum | | | |
| Gellan Gum | 0.05 | 0.05 | 0.05 |
| Xanthan Gum | 0.10 | 0.10 | 0.10 |
| Hydroxypropyl | 0.75 | 0.75 | 0.75 |
| Starch Phosphate | | | |

1.-12. (canceled)

13. An active ingredient combination, wherein the combination comprises (i) one or more alkylamidothiazoles and (ii) one or more cosmetically or dermatologically acceptable biopolymers which are capable for increasing a viscosity of an aqueous solution and are selected from polysaccharides etherified and/or esterified with alcohols having one or more hydroxyl groups and/or with carboxylic acids as well as unetherified and unesterified polysaccharides, the polysaccharides being composed of interlinked glucose and/or mannose and/or galactose and/or glucuronic acid and/or rhamnose units, provided that if xanthan or hydroxypropyl starch phosphate is selected as biopolymer, at least one further biopolymer is present.

14. The active ingredient combination of claim 13, wherein (ii) comprises one or more of dehydroxanthan, gellan gum, xanthan, hydroxypropyl starch phosphate.

15. The active ingredient combination of claim 14, wherein (ii) comprises (a) dehydroxanthan, (b) gellan, (c) xanthan, and (d) hydroxypropyl starch phosphate, weight ratios of (a):(b), (a):(c), (a):(d), (b):(c), (b):(d) and (c):(d) being independently rational numbers of from 0 to 10.

16. The active ingredient combination of claim 14, wherein (ii) comprises (a) dehydroxanthan, (b) gellan, (c) xanthan, and (d) hydroxypropyl starch phosphate, weight ratios (a):(b):(c):(d) in relation to another being (0.1 to 5):(0.1 to 5):(0.1 to 5):(0.1 to 5).

17. The active ingredient combination of claim 13, wherein (i) comprises one or more alkylamidothiazoles of formula 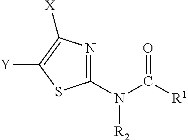
in which
R¹, R², X and Y are different, partially identical or entirely identical and independently have the following meanings:
R¹=—C₁-C₂₄ alkyl (linear and branched), —C₁-C₂₄ alkenyl (linear and branched), —C₁-C₈ cycloalkyl, —C₁-C₈ cycloalkyl-alkylhydroxy, —C₁-C₂₄ alkylhydroxy (linear and branched), —C₁-C₂₄ alkylamine (linear and branched), —C₁-C₂₄ alkylaryl (linear and branched), —C₁-C₂₄ alkylaryl-alkyl-hydroxy (linear and branched), —C₁-C₂₄ alkylheteroaryl (linear and branched), —C₁-C₂₄ alkyl-O—C₁-C₂₄ alkyl (linear and branched), —C₁-C₂₄ alkyl-morpholino, —C₁-C₂₄ alkyl-piperidino, —C₁-C₂₄ alkyl-piperazino, —C₁-C₂₄ alkyl-piperazino-N-alkyl,
R²=H, —C₁-C₂₄ alkyl (linear and branched), —C₁-C₂₄ alkenyl (linear and branched), —C₁-C₈ cycloalkyl, —C₁-C₂₄ hydroxyalkyl (linear and branched), —C₁-C₂₄ alkylaryl (linear and branched), —C₁-C₂₄ alkylheteroaryl (linear and branched),
X=—H, —C₁-C₂₄ alkyl (linear and branched), —C₁-C₂₄ alkenyl (linear and branched), —C₁-C₈ cycloalkyl, —C₁-C₂₄ aryl (optionally mono- or polysubstituted by —OH, —F, —Cl, —Br, —I, —OMe, —NH₂, —CN), —C₁-C₂₄ heteroaryl (optionally mono- or polysubstituted by —OH, —F, —Cl, —Br, —I, —OMe, —NH₂, —CN), —C₁-C₂₄ alkylaryl (linear and branched), —C₁-C₂₄ alkylheteroaryl (linear and branched), -aryl (optionally mono- or polysubstituted by —OH, —F, —Cl, —Br, —I, —OMe, —NH₂, —CN), -phenyl, -2,4-dihydroxyphenyl, -2,3-dihydroxyphenyl, -2,4-dimethoxyphenyl, -2,3-dimethoxyphenyl,
Y=H, —C₁-C₂₄ alkyl (linear and branched), —C₁-C₂₄ alkenyl (linear and branched), —C₁-C₈ cycloalkyl, —C₁-C₂₄ aryl, —C₁-C₂₄ heteroaryl, —C₁-C₂₄ alkylaryl (linear and branched), —C₁-C₂₄ alkylheteroaryl (linear and branched), -aryl, -phenyl, -2,4-dihydroxyphenyl, -2,3-dihydroxyphenyl, -2,4-dimethoxyphenyl, -2,3-dimethoxyphenyl, —COO-alkyl, —COO-alkenyl, —COO-cycloalkyl, —COO-aryl, —COO-heteroaryl,
and X, Y may optionally also=fused aromatic,
where X and Y may form, with one another, aromatic or aliphatic homo- or heterocyclic ring systems having up to n ring-forming atoms, and where the number n may assume values from 5 to 8, and the respective ring systems in turn may be substituted by up to n−1 alkyl groups, hydroxyl groups, carboxyl groups, amino groups, nitrile functions, sulfur-containing substituents, ester groups and/or ether groups,
the alkylamidothiazole(s) being present as either free base or as cosmetically and dermatologically usable salts.

18. The active ingredient combination of claim 13, wherein (i) comprises one or more alkylamidothiazoles of the following formulae: 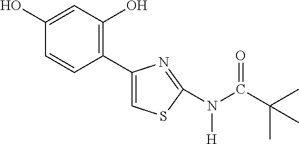 [SMILES: CC(C)(C)C(=O)Nc1nc(-c2ccc(O)cc2O)cs1]
N-(4-(2,4-dihydroxyphenyl)thiazol-2-yl)pivalamide 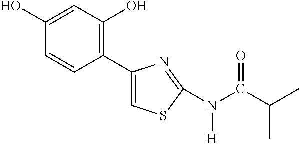 [SMILES: CC(C)C(=O)Nc1nc(-c2ccc(O)cc2O)cs1]
N-(4-(2,4-dihydroxyphenyl)thiazol-2-yl)isobutyramide 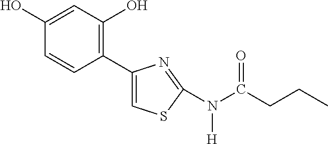 [SMILES: CCCC(=O)Nc1nc(-c2ccc(O)cc2O)cs1]
N-(4-(2,4-dihydroxyphenyl)thiazol-2-yl)butyramide 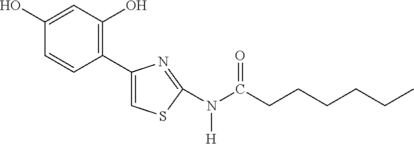 [SMILES: CCCCCCC(=O)Nc1nc(-c2ccc(O)cc2O)cs1]
N-(4-(2,4-dihydroxyphenyl)thiazol-2-yl)heptanamide 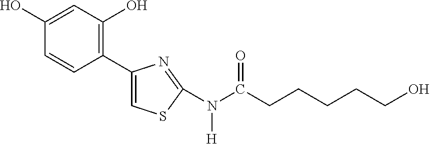 [SMILES: O=C(CCCCCO)Nc1nc(-c2ccc(O)cc2O)cs1]
N-(4-(2,4-dihydroxyphenyl)thiazol-2-yl)-6-hydroxyhexanamide 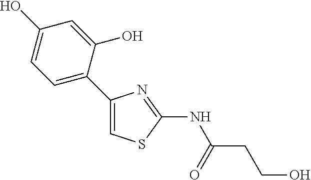 [SMILES: O=C(CCO)Nc1nc(-c2ccc(O)cc2O)cs1]
N-(4-(2,4-dihydroxyphenyl)thiazol-2-yl)-3-hydroxypropanamide 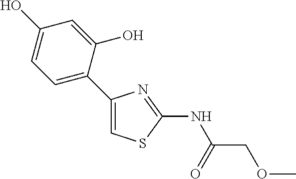 [SMILES: COCC(=O)Nc1nc(-c2ccc(O)cc2O)cs1]
N-(4-(2,4-dihydroxyphenyl)thiazol-2-yl)-2-methoxyacetamide 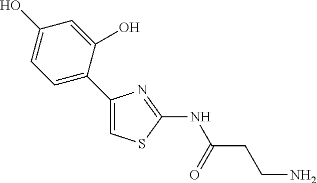 [SMILES: NCCC(=O)Nc1nc(-c2ccc(O)cc2O)cs1]
3-amino-N-(4-(2,4-dihydroxyphenyl)thiazol-2-yl)propanamide 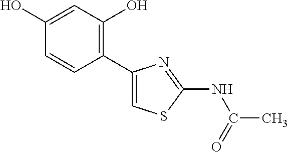 [SMILES: CC(=O)Nc1nc(-c2ccc(O)cc2O)cs1]
N-(4-(2,4-dihydroxyphenyl)thiazol-2-yl)acetamide 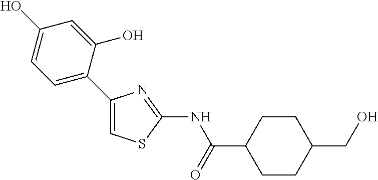 [SMILES: O=C(Nc1nc(-c2ccc(O)cc2O)cs1)C1CCC(CO)CC1]
N-(4-(2,4-dihydroxyphenyl)thiazol-2-yl)-4-(hydroxymethyl)cyclohexanecarboxamide 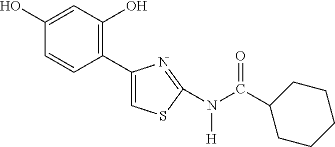 [SMILES: O=C(Nc1nc(-c2ccc(O)cc2O)cs1)C1CCCCC1]
N-(4-(2,4-dihydroxyphenyl)thiazol-2-yl)cyclohexanecarboxamide
and 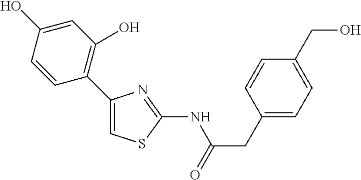 [SMILES: O=C(Cc1ccc(CO)cc1)Nc1nc(-c2ccc(O)cc2O)cs1]
N-(4-(2,4-dihydroxyphenyl)thiazol-2-yl)-2-(4-(hydroxymethyl)phenyl)acetamide

19. The active ingredient combination of claim 13, wherein the one or more alkylamidothiazoles are present as one or more of a halide, carbonate, ascorbate, sulfate, acetate and/or phosphate.

20. A cosmetic or dermatological preparation, wherein the preparation comprises the active ingredient combination of claim 13.

21. The preparation of claim 20, wherein the preparation comprises from 0.000001% to 10% by weight of (i), based on a total weight of the preparation.

22. The preparation of claim 20, wherein the preparation comprises from 0.0001% to 3% by weight of (i), based on a total weight of the preparation.

23. The preparation of claim 20, wherein the preparation comprises from 0.001% to 1% by weight of (i), based on a total weight of the preparation.

24. The preparation of claim 20, wherein the preparation comprises from 0.0001% to 10% by weight of (ii), based on a total weight of the preparation.

25. The preparation of claim 20, wherein the preparation comprises from 0.001% to 5% by weight of (ii), based on a total weight of the preparation.

26. The preparation of claim 20, wherein the preparation comprises from 0.005% to 3% by weight of (ii), based on a total weight of the preparation.

27. The preparation of claim 20, wherein the preparation has a pH of from 7 to 9.

28. The preparation of claim 20, wherein the preparation has a pH of from 7 to 8.

29. The preparation of claim 20, wherein the preparation has a pH of from 7.4 to 7.6.

30. A method for lightening human skin, wherein the method comprises applying to skin the preparation of claim 20 in an amount which is effective for lightening skin.
